(19) United States
(12) Patent Application Publication
(10) Pub. No.: US 2019/0239861 A1
(43) Pub. Date: Aug. 8, 2019

(54) ULTRASONIC DIAGNOSTIC APPARATUS

(71) Applicant: Canon Medical Systems Corporation, Otawara-shi (JP)

(72) Inventors: Yutaka KOBAYASHI, Nasushiobara (JP); Atsushi Nakai, Nasushiobara (JP); Shigemitsu Nakaya, Nasushiobara (JP); Jiro Higuchi, Otawara (JP); Kazuo Tezuka, Nasushiobara (JP); Yoshitaka Mine, Nasushiobara (JP)

(73) Assignee: Canon Medical Systems Corporation, Otawara-shi (JP)

(21) Appl. No.: 16/268,521

(22) Filed: Feb. 6, 2019

(30) Foreign Application Priority Data
Feb. 7, 2018 (JP) 2018-020244
Feb. 4, 2019 (JP) 2019-018207

(51) Int. Cl. A61B 8/00; A61B 8/08; A61B 5/044

(52) U.S. Cl. CPC A61B 8/543; A61B 8/463; A61B 8/488; A61B 5/044; A61B 8/5261

(57) ABSTRACT

According to one embodiment, an ultrasonic diagnostic apparatus includes processing circuitry. The processing circuitry configured to select at least one heartbeat from among a plurality of heartbeats based on a heartbeat selection condition, generate a highlight image in which a range corresponding to the selected heartbeat is emphasized, and display an electrocardiographic waveform corresponding to the heartbeats, an ultrasonic image corresponding to the electrocardiographic waveform, and the highlight image.

### CROSS-REFERENCE TO RELATED APPLICATIONS

This application is based upon and claims the benefit of priority from Japanese Patent Applications No. 2018-020244, filed Feb. 7, 2018; and No. 2019-018207, filed Feb. 4, 2019; the entire contents of both of which are incorporated herein by reference.

### FIELD

Embodiments described herein relate generally to an ultrasonic diagnostic apparatus.

### BACKGROUND

In echocardiography, after a freeze operation, an operator manually selects a heartbeat that seems the most suitable from several closely occurring heartbeats, and thereby various measurements and analyses are executed on an ultrasonic image corresponding to the selected heartbeat. Examples of measurement and analysis methods for an ultrasonic image include two-dimensional wall motion tracking (WMT) and three-dimensional WMT, which are to analyze myocardial wall motions, and automated ejection fraction (auto EF), which is to automatically calculate the left-heart ejection fraction.

In such echocardiography, the operator, who may be a laboratory technician or surgeon, basically makes a visual observation of several heartbeats, and subjectively determines and selects a heartbeat that seems suitable for measurement and analysis. In the case of a patient who does not have an irregular heartbeat, the result of the measurement and analysis executed on the ultrasonic image corresponding to the heartbeat selected by the operator would not significantly differ from the result of the measurement and analysis executed on an ultrasonic image corresponding to a randomly extracted heartbeat, and thus would not cause a problem. For instance, no considerable error occurs in the calculation result of volume information from the auto EF, such as the end-diastolic volume (EDV), end-systolic volume (ESV), and ejection fraction (EF).

In contrast, in the case of a patient who has an irregular heartbeat such as atrial fibrillation, irregular heartbeats may cause a considerable error in the analysis and measurement results of an ultrasonic image of a heartbeat, depending on a selected heartbeat.

The operator may select a heartbeat that is not suitable for the measurement and analysis of an ultrasonic image. If this is the case, the operator who has executed the measurement and analysis on an ultrasonic image corresponding to the selected heartbeat needs to re-select another heartbeat and re-execute the measurement and analysis on the ultrasonic image corresponding to this heartbeat. In addition, the operator may select the heartbeats by trial and error, which may take an unnecessarily long time to select a heartbeat before starting the measurement and analysis. The technique of an ultrasonic diagnostic apparatus is therefore yet to be improved from the aspect of examination efficiency.

### BRIEF DESCRIPTION OF THE DRAWINGS

FIG. 1 is a diagram illustrating the configuration of an ultrasonic diagnostic apparatus according to the first embodiment.

FIG. 2 is a flowchart of the operation of control circuitry when the ultrasonic diagnostic apparatus according to the first embodiment selects a desired heartbeat.

FIG. 3 is a histogram displayed on the monitor by the ultrasonic diagnostic apparatus according to the first embodiment.

FIG. 4 is a diagram explaining the method of the ultrasonic diagnostic apparatus generating histogram data of heartbeats according to the first embodiment.

FIG. 5 is a diagram explaining another example of the method of the ultrasonic diagnostic apparatus generating the histogram data of heartbeats according to the first embodiment.

FIG. 6 is a diagram representing heartbeats displayed on the monitor by the ultrasonic diagnostic apparatus according to the first embodiment.

FIG. 7 is a diagram illustrating the configuration of an ultrasonic diagnostic apparatus according to the second embodiment.

FIG. 8 is a flowchart of the operation of the control circuitry when the ultrasonic diagnostic apparatus according to the second embodiment selects a desired heartbeat.

FIG. 9 is a diagram showing heartbeats displayed on the monitor by the ultrasonic diagnostic apparatus according to the second embodiment.

FIG. 10 is a flowchart of the operation of the control circuitry when the ultrasonic diagnostic apparatus according to a modification example of the second embodiment selects a desired heartbeat.

FIG. 11 is a diagram explaining display modes of heartbeats displayed on the monitor by an ultrasonic diagnostic apparatus according to another embodiment.

### DETAILED DESCRIPTION

In general, according to one embodiment, an ultrasonic diagnostic apparatus includes processing circuitry. The processing circuitry configured to select at least one heartbeat from among a plurality of heartbeats based on a heartbeat selection condition, generate a highlight image in which a range corresponding to the selected heartbeat is emphasized, and display an electrocardiographic waveform corresponding to the heartbeats, an ultrasonic image corresponding to the electrocardiographic waveform, and the highlight image.

The embodiments will be explained by referring to the drawings.

### First Embodiment

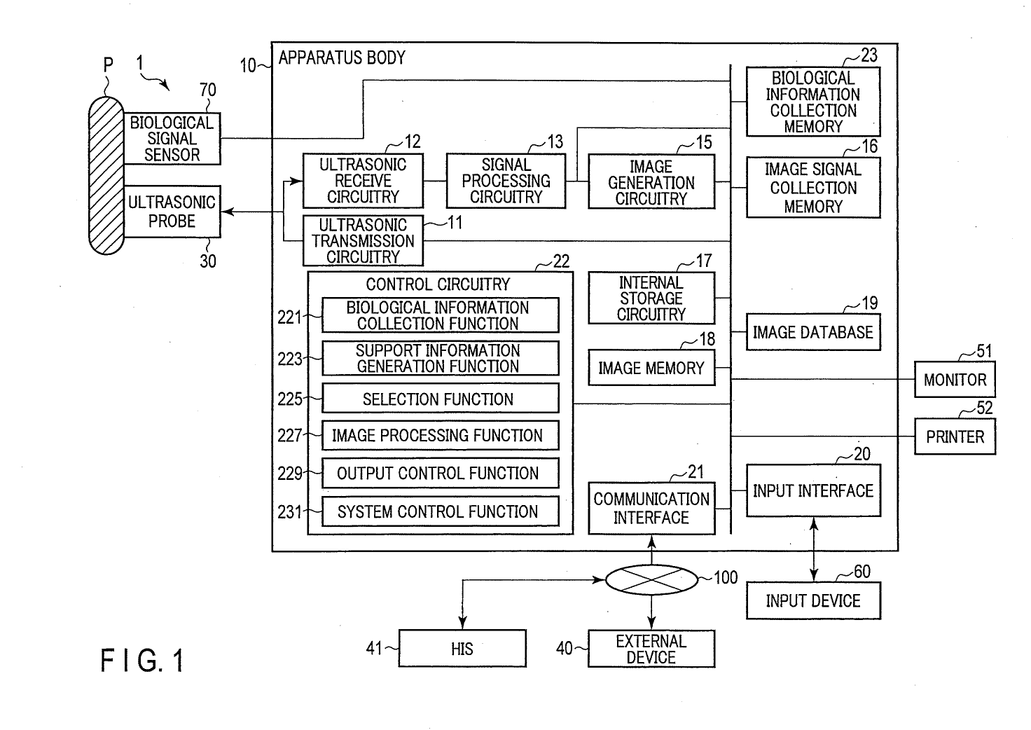

The ultrasonic diagnostic apparatus 1 according to the first embodiment will be explained with reference to the block diagram of FIG. 1.

As illustrated in FIG. 1, an ultrasonic diagnostic apparatus 1 comprises an apparatus body 10, an ultrasonic probe 30, a monitor 51, a printer 52, an input device 60, and a biological signal sensor 70. The apparatus body 10 is coupled to an external device 40, a hospital information system (HIS) 41, and the like via a network 500. The apparatus body 10 is also coupled to the monitor 51, the printer 52, and the input device 60.

The ultrasonic probe 30 includes a plurality of piezoelectric oscillators, a matching layer deposited on the piezoelectric oscillators, and a backing material for preventing the ultrasonic waves from propagating backward from the piezoelectric oscillators, and the like. The ultrasonic probe 30 is detachably coupled to the apparatus body 10. The piezoelectric oscillators generate ultrasonic waves in response to drive signals supplied from ultrasonic transmission circuitry 11 of the apparatus body 10. The ultrasonic probe 30 may be provided with buttons that are to be pressed for offset processing and ultrasonic image freezing, which will be discussed later. Here, freezing denotes a mode in which no ultrasonic image is being collected.

When an ultrasonic wave is transmitted from the ultrasonic probe 30 to a subject P, the transmitted ultrasonic wave is sequentially reflected on the acoustic impedance discontinuity surfaces in the living tissue of the subject P. The ultrasonic wave reflected in the living tissue is received as a reflection wave (echo) by the piezoelectric oscillators of the ultrasonic probe 30. The amplitude of a reception signal generated from the received reflection wave depends on the difference in acoustic impedances at the discontinuity surface on which the ultrasonic wave is reflected. If the transmitted ultrasonic pulse is reflected in a flowing bloodstream, or on the surface of the moving cardiac wall or the like, the frequency of the reflection wave signal is shifted depending on the velocity components of the moving object in the direction of transmitting ultrasonic waves due to the Doppler effect. The ultrasonic probe 30 receives the reflection wave from the subject P, and converts the reflection wave into an electrical signal (reception signal). In this manner, a reception signal is generated. The reception signal may also be referred to as a reflection wave signal. The ultrasonic probe 30 may be a 1D array probe in which piezoelectric transducers are aligned in a predetermined direction, a 2D array probe in which piezoelectric transducers are aligned in a two-dimensional matrix form, or a mechanical 4D probe that realizes an ultrasonic scan by mechanically sweeping the piezoelectric transducers in a direction orthogonal to the alignment direction of the piezoelectric oscillators.

The biological signal sensor 70 detects a biological signal from the subject P subjected to the ultrasonic wave scanning. The biological signal sensor 70 may detect an electrocardiogram (ECG) signal of the subject P as an electric signal. After executing various processes including digitization processing onto the detected ECG signal, the biological signal sensor 70 suitably transmits this signal as heartbeat data to the circuits of the apparatus body 10. The biological signal sensor 70 may detect other biological signals having periodicity that arise from the subject P, such as brain waves, pulses, and respiration.

The apparatus body 10 in FIG. 1 is a device that generates ultrasonic images in accordance with the reception signals received by the ultrasonic probe 30. As shown in FIG. 1, the apparatus body 10 includes ultrasonic transmission circuitry 11, ultrasonic receive circuitry 12, signal processing circuitry 13, image generation circuitry 15, an image signal collection memory 16, internal storage circuitry 17, an image memory 18, an image database 19, an input interface 20, a communication interface 21, control circuitry 22, and a biological information collection memory 23.

The ultrasonic transmission circuitry 11 is a processor that supplies a drive signal to the ultrasonic probe 30. The ultrasonic transmission circuitry 11 is realized, for example, by trigger generation circuitry, delay circuitry, and pulser circuitry. The trigger generation circuitry repeats to generate rate pulses at a certain rate frequency under the control of the control circuitry 22 so as to form transmission waves. The delay circuitry provides individual rate pulses generated by the trigger generation circuitry, with a delay time for each piezoelectric oscillator. This delay time is necessary to converge the ultrasonic wave generated by the ultrasonic probe 30 into a beam and thereby determine the transmission directivity. The pulser circuitry applies a drive signal (drive pulse) to the ultrasonic probe 30 at the timing of a rate pulse under the control of the control circuitry 22. By changing the delay time to be applied to each rate pulse from the delay circuitry, the transmission direction from the ultrasonic transducer surface can be discretionarily adjusted.

The ultrasonic receive circuitry 12 is a processor that executes various processes on the reception signal received by the ultrasonic probe 30. The ultrasonic receive circuitry 12 may be realized by amplifier circuitry, A/D converter, reception delay circuitry, and adder. The amplifier circuitry executes gain correction processing by amplifying the reception signal received by the ultrasonic probe 30 for each channel. The A/D converter converts the gain-corrected reception signal to a digital signal. The reception delay circuitry provides the digital signal with a delay time necessary to determine the reception directivity. The adder adds a plurality of digital signals to which a delay time is given. With the addition processing by the adder, a reception signal is generated in which a reflection component from a direction corresponding to the reception directivity is emphasized. Such a reception signal may include amplitude information in which the difference between the acoustic impedances of the tissue is incorporated, and phase information in which the motion of the biological tissue such as movement or traveling velocity is incorporated.

The signal processing circuitry 13 is a processor that executes various processes on the reception signal received from the ultrasonic receive circuitry 12. The signal processing circuitry 13 executes envelop detection processing and logarithmic amplification processing on the reception signal received from the ultrasonic receive circuitry 12, thereby generating data in which the signal intensity is expressed by the brightness (B-mode data). The generated B-mode data is stored as B-mode raw data on a two-dimensional ultrasonic scan line in a raw data memory (not shown).

Also, the signal processing circuitry 13 executes a frequency analysis on the reception signal received from the ultrasonic receive circuitry 12 to extract a bloodstream signal, and generates data (Doppler data) by extracting information such as the average velocity, distribution, and power with regard to multiple points from the bloodstream signal. The generated Doppler data is stored as two-dimensional. Doppler raw data of the ultrasonic scan line in the not-shown raw data memory.

The signal processing circuitry 13 extracts a bloodstream signal from the reception signal received from the ultrasonic receive circuitry 12, and generates, from the extracted bloodstream signal, Doppler spectrum image data that represents a Doppler spectrum image indicating a Doppler waveform. The Doppler waveform may be a waveform of the blood flow velocity plotted in chronological order in a range defined as an observation target site. That is, the Doppler waveform represents temporal variation of the blood flow velocity.

The image generation circuitry 15 is a processor configured to generate various types of ultrasonic image data, based on the data generated by the signal processing circuitry 13.

The image generation circuitry 15 generates B-mode image data, based on the B-mode raw data stored in the raw data memory. The B-mode image based on the B-mode image data may show objects in the subject P. The B-mode image data has pixel values (brightness values) in which, for example, the characteristics of the ultrasound probe such as ultrasound wave convergence and the acoustic field characteristics of ultrasound beams (e.g., transmission/reception beams) are incorporated. For example, the B-mode image data exhibits a relatively high brightness in and around the in-focus region of the ultrasound wave in the scanned area than in the out-of-focus region of the ultrasound. The image generation circuitry 15 may store the generated B-mode image data in the image signal collection memory 16, in association with the heartbeat data output from the biological signal sensor 70.

The image generation circuitry 15 generates Doppler image data that represents moving object information, based on the Doppler raw data stored in the raw data memory. The Doppler image data may be velocity image data, distribution image data, or power image data, or a combination of the above. The image generation circuitry 15 stores the generated Doppler image data, for example in the image signal collection memory 16, in association with the heartbeat data output from the biological signal sensor 70.

Using a conventional technique, the image generation circuitry 15 converts (scan-converts) the scan line signal string of the ultrasonic scanning to a scan line signal string of a video format typified by television, and thereby generates the ultrasonic image data for display. Specifically, the image generation circuitry 15 executes coordinate conversion in accordance with the ultrasonic scanning mode of the ultrasonic probe 30 to generate the to-be-displayed ultrasonic image data. The image generation circuitry 15 stores the generated ultrasonic image data, in association with the heartbeat data output from the biological signal sensor 70, in the image signal collection memory 16. The ultrasonic image based on the to-be-displayed ultrasonic image data may be displayed on the monitor 51. For the monitor 51, a device such as a CRT display, liquid crystal display, organic EL display, LED display, plasma display, or any other display known in the field of the technology may be suitably adopted. The ultrasonic image based on the to-be-displayed ultrasonic image data is printed out by the printer 52 in accordance with a preset format.

The image generation circuitry 15 may further execute various processes including corrections to the dynamic range, brightness, contrast, and y curve, as well as an RGB conversion, on the generated ultrasonic image data of various types. The image generation circuitry 15 may also add to the generated ultrasonic image data, text information for various parameters, and additional information such as scale markings and body mark.

The image generation circuitry 15 may generate a user interface (Graphical User Interface or GUI) through which the operator (e.g., laboratory technician and surgeon) can enter various commands on the input interface 20, and display the GUI on the monitor 51.

The image signal collection memory 16 includes, for example, a magnetic or optical storage medium, or a storage medium that can be read by a processor such as a semiconductor memory. The image signal collection memory 16 stores therein various types of ultrasonic image data generated by the image generation circuitry 15. The image signal collection memory 16 stores image data corresponding to a plurality of frames that are entered immediately before the freeze operation via the input interface 20.

The image signal collection memory 16 stores the ultrasonic image data associated with the heartbeat data, for a plurality of heartbeats. The image data stored in the image signal collection memory 16 is associated with the heartbeat data of the subject P for every heartbeat (one cardiac cycle). Specifically, each item of the image data stored in the image signal collection memory 16 is associated with the heartbeat data for one heartbeat. The image signal collection memory 16 may store the ultrasonic image data for one heartbeat as one item of image data, or the ultrasonic image data for a plurality of heartbeats as one item of image data.

The image data stored in the image signal collection memory 16 may be sequentially displayed (cine-playback). The image data stored in the image signal collection memory 16 may be the image data representing an image that is to be displayed on the monitor 51. Such an image may include images based on the ultrasonic image data acquired by ultrasonic scanning, as well as images based on medical image data acquired by other medical image diagnostic apparatus such as CT image data, MR image data, X-ray image data, and PET image data.

The image signal collection memory 16 may also store the data generated by the signal processing circuitry 13. The B-mode data or Doppler data stored in the image signal collection memory 16 may be retrieved by the operator after the examination. Such data is processed at the image generation circuitry 15 into to-be-displayed ultrasonic image data.

The internal storage circuitry 17 includes, for example, a magnetic or optical storage medium, or a storage medium that can be read by a processor such as a semiconductor memory. The internal storage circuitry 17 stores therein control programs for realizing ultrasonic transmission/reception, for image processing, and for display processing. The internal storage circuitry 17 further stores a control program for realizing various functions according to the present embodiment. In addition, the internal storage circuitry 17 stores groups of data such as diagnostic information (including patient IDs and medical opinions), diagnostic protocols, a body mark generation program, and a conversion table that presets the range of visualization color data for respective diagnostic sites. The internal storage circuitry 17 may also store anatomical charts, such as an atlas, of the structure of organs of a living body.

The internal storage circuitry 17 further stores therein various ultrasonic image data generated by the image generation circuitry 15 in accordance with a storage operation that is entered through the input interface 20. The internal storage circuitry 17 may store therein items of ultrasonic image data generated by the image generation circuitry 15 in accordance with the storage operation entered through the input interface 20 in such a manner as to include the operation order and operation time. The internal storage circuitry 17 may be configured to transfer the stored data to an external device via the communication interface 21.

The image memory 18 includes, for example, a magnetic or optical storage medium, or a storage medium that can be read by a processor such as a semiconductor memory. The image memory 18 stores, of the ultrasonic image data for a plurality of heartbeats stored in the image signal collection memory 16, ultrasonic image data for at least one heartbeat corresponding to the collected heartbeat data.

The image database 19 may store the image data transferred from the external device 40. For example, the image database 19 acquires from the external device 40 previous image data acquired in previous examinations for the same patient, and stores this data. The previous image data may include ultrasonic image data, computed tomography (CT) image data, magnetic resonance (MR) image data, positron emission tomography-computed tomography (PET-CT) image data, PET-MR image data, and X-ray image data. The previous image data may be stored as volume data or rendering image data.

The image database 19 may load image data from a storage media such as a MO, CD-R, and DVD to store desired image data.

The input interface 20 receives various commands and information from the operator through the input device 60. The input device 60 may include a mouse, a keyboard, panel switches, slider switches, dial switches, a track ball, a rotary encoder, an operation panel, a touch command screen (TCS), and the like. The input device 60 may further include a group of switches for switching imaging modes including ultrasonic transmission/reception scheme and reception signal processing scheme. The group of switches are not limited to mechanical devices such as dial switches and/or a track ball, but may be an operation panel image displayed on the TCS, or an operation panel image displayed on the second console of the external device 40.

The input interface 20 is coupled to the control circuitry 22 via a bus, and the input interface 20 thereby converts an operation command entered by the operator to an electric signal and outputs the electric signal to the control circuitry 22. Throughout the specification, the input interface 20 is not limited to a component coupled to a physical operation component such as a mouse and keyboard. Examples of the input interface 20 may include electric signal processing circuitry configured to receive, as a wireless signal, an electric signal corresponding to an operation command that is entered from an external input device provided separately from the ultrasonic diagnostic apparatus 1 and to output this electric signal to the control circuitry 22.

The communication interface 21 is coupled to the external device 40 via the network 500 to perform data communications with the external device 40. The external device 40 may be a database such as Picture Archiving and Communication System (PACS), which is a system for managing various medical image data. The external device 40 may be any medical image diagnostic apparatus other than the ultrasonic diagnostic apparatus 1 of the present embodiment, such as an X-ray CT apparatus, magnetic resonance imaging (MRI) apparatus, nuclear medicine diagnostic apparatus, and X-ray diagnostic apparatus. For communications with the external device 40, any communication standards may be adopted, examples of which include DICOM.

The communication interface 21 is coupled to the HIS 41 via the network 500 and performs data communications with the HIS 41. The HIS 41 may include an examination order issuance system for issuing examination order information to request an examination, and an electronic chart system for managing electronic charts to which medical images are attached. The communication interface 21 receives examination order information from the HIS 41. For the communications with the HIS 41, any communication standard may be adopted, examples of which include Health Level 7 (HL7).

The control circuitry 22 may be a processor that serves as the center of the ultrasonic diagnostic apparatus 1. The control circuitry 22 executes an operation program stored in the internal storage circuitry 17 and thereby realizes functions corresponding to this operation program. Specifically, the control circuitry 22 includes a biological information collection function 221, a support information generation function 223, a selection function 225, an image processing function 227, an output control function 229, and a system control function 231.

The biological information collection function 221 is to collect biological information output from the biological signal sensor 70. When the biological information collection function 221 is implemented, the control circuitry 22 collects, for example, heartbeat data output from the biological signal sensor 70, and stores the data in the biological information collection memory 23 in association with various types of ultrasonic image data generated by the image generation circuitry 15. Alternatively, with the biological information collection function 221, the control circuitry 22 acquires heartbeat data of a plurality of heartbeats of the patient for a duration of time for acquiring the ultrasonic images.

The support information generation function 223 is to generate support information for supporting the selection of a desired heartbeat. The “certain heartbeat” may be a heartbeat designated through the input interface 20, or a heartbeat selected by the ultrasonic diagnostic apparatus 1. When the support information generation function 223 is implemented, the control circuitry 22 generates histogram data representing a heartbeat histogram showing the relationship between the length of one heartbeat and the number of heartbeats counted according to each specific length, using the heartbeat data corresponding to the heartbeats. The horizontal axis of the heartbeat histogram indicates the length of one heartbeat. The vertical axis of the heartbeat histogram indicates the number (frequency) of heartbeats having a specific length among the heartbeats included in the heartbeat data corresponding to a specific measurement period. Specifically, the control circuitry 22 calculates the length of each of the heartbeats included in the heartbeat data corresponding to the specific measurement period stored in the biological information collection memory 23. The control circuitry 22 counts the number of heartbeats for the length of each of the calculated heartbeats, and generates the heartbeat histogram data. Here, the length of each calculation-targeted heartbeat is allowed for a certain latitude with respect to a reference length. When the reference length is X (msec) (X>0), heartbeats for a certain calculation target may have lengths between X (msec) and X+Y (msec) (Y>0).

The support information is not limited to the above histogram. The support information may be an electrocardiographic waveform in which certain heartbeats are highlighted. Specifically, the support information includes at least one of “an electrocardiographic waveform in which any consecutive heartbeats that exhibit the ratio of their lengths of heartbeats being approximately 1 are highlighted”, “an electrocardiographic waveform in which heartbeats having approximately the same length as the length of a heartbeat based on a heart rate counted in the previous ultrasonic examination are highlighted”, “an electrocardiographic waveform in which heartbeats having approximately the same length as the length of a heartbeat based on a heart rate counted at an examination using a different medical image diagnostic apparatus are highlighted” and “an electrocardiographic waveform in which heartbeats having approximately the same length as the designated length are highlighted”.

With the support information generation function 223, the control circuitry 22 generates highlight image data representing a highlight image to be displayed on the monitor 51 in which certain heartbeats are emphasized. A highlight image includes certain heartbeats highlighted to be displayed on the monitor 51. That is, the control circuitry 22 generates a highlight image that includes the ranges of the selected heartbeats being emphasized.

The selection function 225 is to select a heartbeat to be used for the measurement and analysis of an ultrasonic image, using the support information. When the selection function 225 is implemented, the control circuitry 22 may select a heartbeat in the heartbeat histogram, based on the number of heartbeats counted for the same length. Specifically, the control circuitry 22 selects a heartbeat of a length having the highest number of counts.

The image processing function 227 is to extract ultrasonic image data corresponding to the selected heartbeat. When the image processing function 227 is implemented, the control circuitry 22 extracts the ultrasonic image data corresponding to the selected heartbeat, from the ultrasonic image data of a plurality of heartbeats stored in the image signal collection memory 16, and stores the data in the image memory 18. When an image of one image data item contains ultrasonic images of a plurality of heartbeats, the control circuitry 22 may clip from this image an ultrasonic image corresponding to the selected heartbeat, and may store the ultrasonic image data representing the clipped-out ultrasonic image in the image memory 18 as new ultrasonic image data.

The output control function 229 is to output the biological information, support information, ultrasonic image data, and the like. When the output control function 229 is implemented, the control circuitry 22 controls the monitor 51 to display a heartbeat histogram. The control circuitry 22 also controls the monitor 51 to emphasize the selected heartbeat. The control circuitry 22 further controls the monitor 51 to display an electrocardiographic waveform showing heartbeats. That is, the control circuitry 22 displays the support information, the electrocardiographic waveform based on the heartbeat data, and the ultrasonic image corresponding to the electrocardiographic waveform. Alternatively, the control circuitry 22 displays an electrocardiographic waveform showing the selected heartbeat, an ultrasonic image corresponding to the electrocardiographic waveform, and a highlight image emphasizing the range of the selected heartbeats. The control circuitry 22 may control the printer 52 to print out the heartbeat histogram and the electrocardiogram in which the selected heartbeats are emphasized.

The system control function 231 is to control the basic operations of the ultrasonic diagnostic apparatus 1 such as input/output and ultrasonic transmission/reception. When the system control function 231 is implemented, the control circuitry 22 receives the designation of an imaging mode for ultrasonic scanning via the input interface 20. The control circuitry 22 controls the ultrasonic transmission circuitry 11 and the ultrasonic receive circuitry 12 based on the designated imaging mode to implement the ultrasonic scanning. The control circuitry 22 implements the measurement and analysis of the ultrasonic image using various types of ultrasonic images corresponding to the selected heartbeat.

The biological information collection function 221, support information generation function 223, selection function 225, image processing function 227, output control function 229, and system control function 231 may be incorporated as a control program, or hardware circuitry dedicated to respective functions may be incorporated in the control circuitry 22 or the apparatus body 10 as circuitry that the control circuitry 22 can refer to.

The biological information collection memory 23 includes, for example, a magnetic or optical storage medium, or a storage medium that can be read by a processor such as a semiconductor memory. The biological information collection memory 23 stores therein biological information output from the biological signal sensor 70, such as heartbeat data. The heartbeat data stored in the biological information collection memory 23 is associated with the image data stored in the image signal collection memory 16 for every heartbeat.

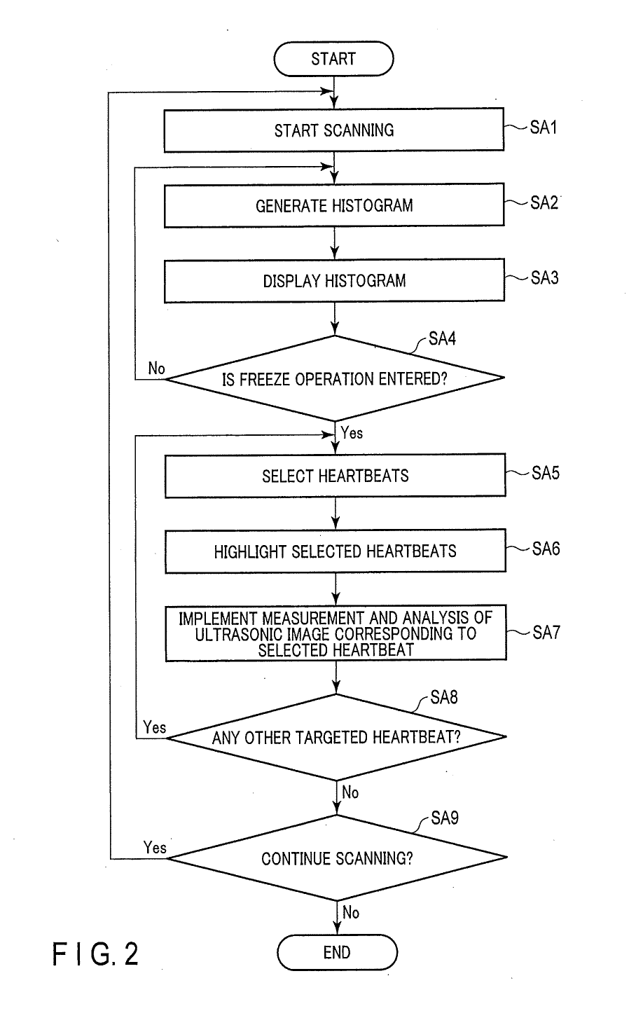

Next, the operation of the ultrasonic diagnostic apparatus 1 according to the first embodiment will be explained with reference to the drawings. FIG. 2 is a flowchart of the operation of the control circuitry 22 when the ultrasonic diagnostic apparatus 1 according to the first embodiment selects a desired heartbeat. In the following explanation, the ultrasonic diagnostic apparatus 1 receives examination order information from the HIS 41, and implements the ultrasonic scanning based on the received examination order information. The examination order information may be entered directly from the ultrasonic diagnostic apparatus 1 via the input interface 20. In accordance with the ultrasonic scanning, the control circuitry 22 collects heartbeat data that is output from the biological signal sensor 70. Here, the control circuitry 22 associates the heartbeat data for every heartbeat with the respective items of the ultrasonic image data generated by the image generation circuitry 15, and sequentially stores the resultant data in the biological information collection memory 23. The control circuitry 22 selects, of the heartbeats having certain lengths and counted when generating the histogram data, two heartbeat having lengths that are most frequently counted as heartbeats for the ultrasonic image measurement and analysis.

When implementing the ultrasonic scanning, a continuous wave (CW) mode, in which continuous waves are transmitted and received to generate Doppler spectrum image data corresponding to one scan line, is adopted as an imaging mode. The imaging mode, however, may be any other mode such as B mode, color Doppler mode, pulsed wave (PW) mode, or M mode. In the B mode, B-mode image data is generated by B-mode scanning. In the color Doppler mode, color Doppler image data, in which, for example, colors are assigned to the bloodstream information that is collected using pulse waves, is generated by color Doppler-mode scanning. The color Doppler-mode scanning includes B-mode scanning. In the PW mode, pulse waves are transmitted and received with respect to the scan line, and Doppler spectrum image data is thereby generated for a specific measurement site. In the M-mode, brightness images on a specific line generated from echo data of a targeted scan line are time-sequentially arranged, and M-mode image data is thereby generated.

As illustrated in FIG. 2, when a start command for starting the ultrasonic scanning is entered via the input interface 20 after receiving the examination order information from the HIS 41, the control circuitry 22 controls the ultrasonic transmission circuitry 11 and the ultrasonic receive circuitry 12 to start the ultrasonic scanning (step SA1).

Next, the control circuitry 22 generates heartbeat histogram data, using, for example, the heartbeat data of a certain duration of the measurement that is stored in the biological information collection memory 23 (step SA2).

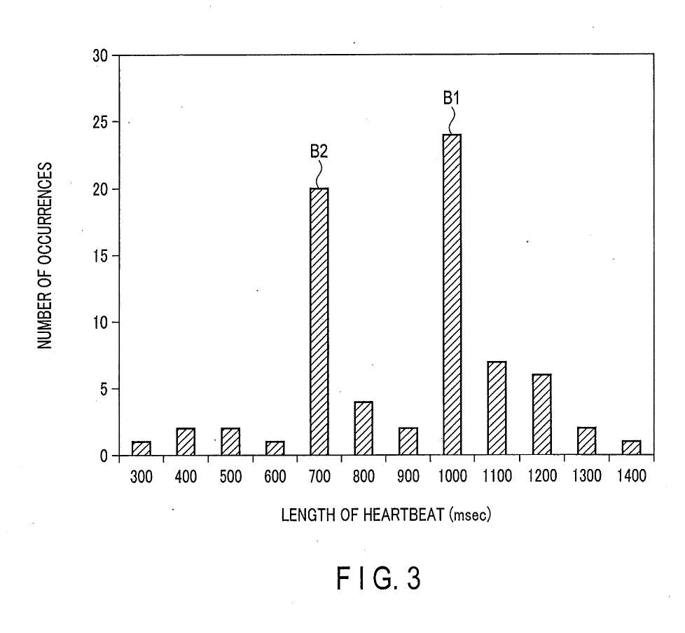

The control circuitry 22 displays a histogram on the monitor 51, based on the generated heartbeat histogram data (step SA3). FIG. 3 shows an example of a histogram displayed on the monitor 51 by the ultrasonic diagnostic apparatus 1 according to the first embodiment. The histogram of FIG. 3 is a graph created by counting the number of heartbeats according to heartbeat lengths. The horizontal axis of the histogram denotes a heartbeat length (msec). The figures indicated along the horizontal axis of the histogram are standard values of the heartbeat lengths. For instance, the figure X in FIG. 3 (X=300, 400, 500, 600, 700, 800, 1000, 1100, 1200, 1300, and 1400) indicates that the length of one heartbeat ranges from X (msec) to X+99 (msec). The vertical axis of the histogram denotes the number of heartbeats for different heartbeat lengths with reference to their standard values. The figure X in FIG. 3 may be defined as one heartbeat having the length of X−49 (msec) to X+50 (msec).

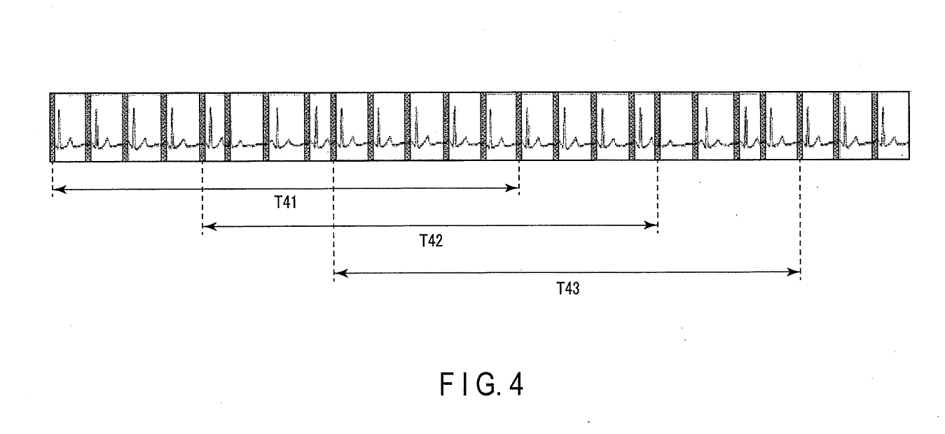

The control circuitry 22 determines whether or not a freeze operation is entered (step SA4). When determining that no freeze operation is entered (no at step SA4), the control circuitry 22 changes the target duration for generating the histogram data for the collected heartbeat data, and repeats the operations at steps SA2 and SA3. The control circuitry 22 thereby updates the histogram displayed on the monitor 51 at predetermined intervals until a freeze operation is entered. FIG. 4 is a diagram explaining the method of the ultrasonic diagnostic apparatus 1 according to the first embodiment generating histogram data of heartbeats. An electrocardiographic waveform of the heartbeat data corresponding to the predefined duration of measurement is shown in FIG. 4. According to FIG. 4, the control circuitry 22 changes the target duration for counting heartbeats from T41 to T42 and to T43 so as to generate the histogram data using a moving average. Here, the length of durations T41, T42, and T43 are set equal to each other. In this manner, the histogram data of the latest heartbeat data can be generated.

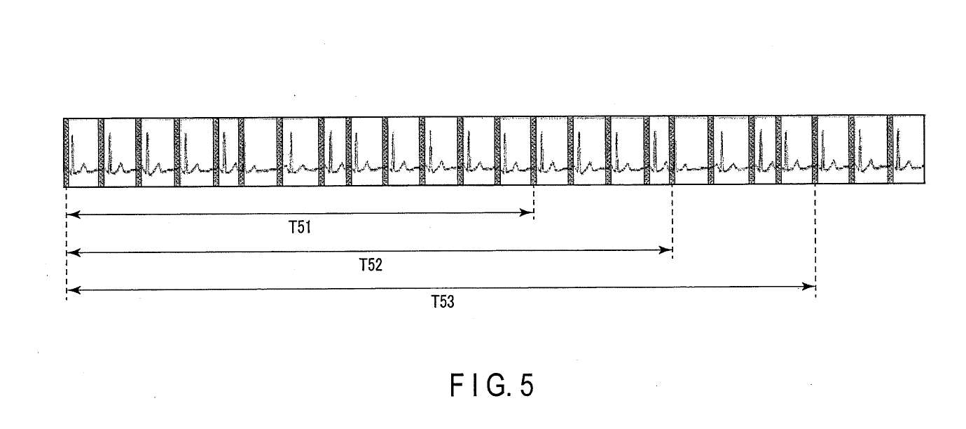

The control circuitry 22 may gradually increase the length of the duration for generating the histogram data with regard to the collected heartbeat data. FIG. 5 is a diagram explaining another example of the method of the ultrasonic diagnostic apparatus generating histogram data of heartbeats according to the first embodiment. An electrocardiographic waveform of the heartbeat data corresponding to a predefined measurement duration is shown in FIG. 5. According to FIG. 5, the control circuitry 22 generates the histogram data while gradually increasing the target duration for counting the heartbeats, from T51 to T52 to T53. In this manner, the number of samples gradually increases, and therefore more reliable histogram data can be generated.

When determining that a freeze operation is entered (yes at step SA4), the control circuitry 22 may select, based on the histogram data generated immediately before this determination, heartbeats to be used for the ultrasonic image measurement and analysis (step SA5). Specifically, the control circuitry 22 may select, of the heartbeats in the histogram of FIG. 3, a plurality of heartbeats having a length which is most frequently counted. In the histogram of FIG. 3, the heartbeats of the most frequently counted length are heartbeats having lengths corresponding to bar B1, which range between 1000 and 1099 (msec).

The control circuitry 22 may allow the operator to designate a heartbeat for the ultrasonic image measurement and analysis from the histogram displayed on the monitor 51. If this is the case, the operator may view the histogram of FIG. 3, and enters a value for the heartbeats corresponding to the bar B1 of FIG. 3 via the input interface 20. When the value for heartbeats is entered, the control circuitry 22 displays an electrocardiographic waveform in which the corresponding heartbeats are highlighted. The operator thereby selects a heartbeat as desired from the displayed electrocardiographic waveform.

Alternatively, when the displayed histogram is based on the GUI for selecting heartbeats, the operator may select the bar B1 via the input interface 20. When the bar B1 is selected from the histogram, the control circuitry 22 displays an electrocardiographic waveform in which the heartbeats corresponding to the selected bar B1 are highlighted. The operator can thereby select any heartbeat from the displayed electrocardiographic waveform. The control circuitry 22 may be configured to automatically select any heartbeat from the electrocardiographic waveform in which certain heartbeats are highlighted.

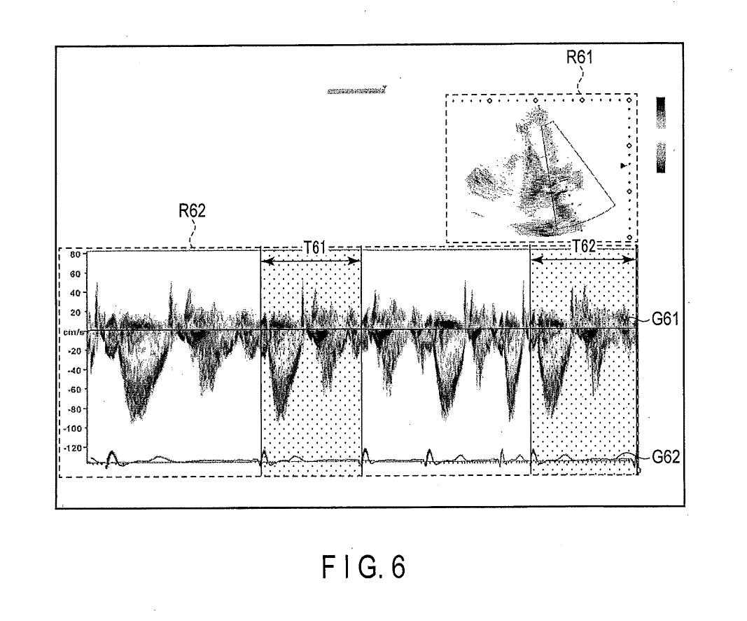

Next, the control circuitry 22 displays the selected heartbeats together with the ultrasonic images corresponding to these heartbeats. Here, the control circuitry 22 highlights the selected heartbeats (step SA6). Specifically, the control circuitry 22 generates highlight image data that represents a highlight image in which the selected heartbeats are highlighted. On the monitor 51, the control circuitry 22 superimposes and displays a highlight image based on the generated highlight image data, onto the selected heartbeats. FIG. 6 is a diagram showing example heartbeats displayed on the monitor 51 by the ultrasonic diagnostic apparatus 1 according to the first embodiment. In FIG. 6, the monitor 51 highlights and displays heartbeats corresponding to the two heartbeats selected at step SA5. The heartbeats of FIG. 6 are displayed in the form of an electrocardiographic waveform. According to FIG. 6, a B-mode image region R61 and a Doppler image region R62 are displayed on the monitor 51.

As indicated in FIG. 6, the B-mode image region R61 includes a B-mode image to identify the scanning position for the ultrasonic scanning, for example, in the CW mode. The B-mode image region R61 may also include a color Doppler image superimposed on the B-mode image.

As indicated in FIG. 6, the Doppler image region R62 includes a Doppler spectrum image G61 acquired by the ultrasonic scanning. The Doppler image region R62 further includes an electrocardiographic waveform G62 based on the heartbeat data in association with the Doppler spectrum image G61 with respect to the temporal axis. Furthermore, in the Doppler spectrum image G61 and electrocardiographic waveform G62, highlight images are superimposed onto the regions included in durations T61 and T62 respectively corresponding to the selected two heartbeats. That is, the control circuitry 22 displays the highlight image for highlighting the regions corresponding to the selected heartbeats, onto the electrocardiographic waveform corresponding to the selected heartbeat and the Doppler spectrum image corresponding to this electrocardiographic waveform. In this manner, the regions included in the durations T61 and T62 are highlighted, which allows the operator to quickly identify the heartbeat suitable for the ultrasonic image measurement and analysis.

Next, the control circuitry 22 implements the measurement and analysis of the ultrasonic image corresponding to the heartbeat designated by the operator via the input interface 20, of the heartbeats selected and displayed on the monitor 51 (step SA7). For example, the control circuitry 22 may implement the 2D WMT, 3D WMT, or auto EF. Here, the control circuitry 22 may extract the ultrasonic image data corresponding to the selected heartbeat from the ultrasonic image data of the heartbeats stored in the image signal collection memory 16, and stores the extracted data in the image memory 18. The control circuitry 22 may be configured to automatically implement the measurement and analysis on the ultrasonic image corresponding to the latest heartbeat from among the heartbeats selected at step SA5.

The control circuitry 22 determines whether or not there is any heartbeat to be used for the ultrasonic image measurement and analysis other than the selected heartbeat (step SA8).

When the control circuitry 22 determines that there is a heartbeat to be used for the ultrasonic image measurement and analysis, other than the selected heartbeat (yes at step SA8), the operations from step SA5 to step SA8 are executed on this other heartbeat. At step SA5 the control circuitry 22 may select, of the heartbeats in the histogram of FIG. 3, the heartbeats of the second most frequently counted length, and executes the operations at steps SA6 through SA8. In FIG. 3, the heartbeats of the second most frequently counted length may be of the lengths ranging between 700 and 799 (msec) corresponding to the bar B2.

At step SA8, the control circuitry 22 determines that no heartbeat other than the selected heartbeats is included for the ultrasonic image measurement and analysis (no at step SA8). The control circuitry 22 therefore determines whether the ultrasonic scanning should be continued (step SA9).

When determining that the ultrasonic scanning should be continued in the absence of a termination command for terminating the ultrasonic scanning (yes at step SA9), the control circuitry 22 re-executes the operations at steps SA1 through SA9.

When a termination command to terminate the ultrasonic scanning is received, the control circuitry 22 determines that the ultrasonic scanning should not be continued (no at step SA9), and terminates the ultrasonic scanning.

According to the first embodiment, the control circuitry 22 collects the heartbeat data output from the biological signal sensor 70 in accordance with the ultrasonic scanning. The control circuitry 22 generates the heartbeat histogram data, using the heartbeat data collected during the predefined measurement duration. The control circuitry 22 displays a histogram on the monitor 51, based on the generated heartbeat histogram data.

In this manner, the operator can select a heartbeat while viewing the histogram displayed on the monitor 51. This reduces the trial and error time for the operator to select a target heartbeat.

Thus, the examination efficiency can be enhanced.

Furthermore, according to the first embodiment, the control circuitry 22 selects a heartbeat for the ultrasonic image measurement and analysis, based on the heartbeat histogram data. The control circuitry 22 generates highlight image data that represents a highlight image in which the selected heartbeats are emphasized. The control circuitry 22 superimposes a highlight image based on the generated highlight image data, onto the selected heartbeats, and displays the resultant image on the monitor 51.

In this manner, the operator can quickly find a heartbeat for the ultrasonic image measurement and analysis based on the heartbeat data. Thus, the trial and error time for the operator to select a target heartbeat can be reduced. Furthermore, because the selection is based on an objective selection condition, erroneous selection of heartbeat data can be avoided. In addition, consistency with the previous examination and consistency that is not affected by different operators can be maintained.

### Second Embodiment

In the first embodiment, the selection of a heartbeat for the ultrasonic image measurement and analysis based on the heartbeat histogram data has been explained. In the second embodiment, the selection of a heartbeat for the ultrasonic image measurement and analysis based on the ratio of the lengths of consecutive heartbeats will be discussed.

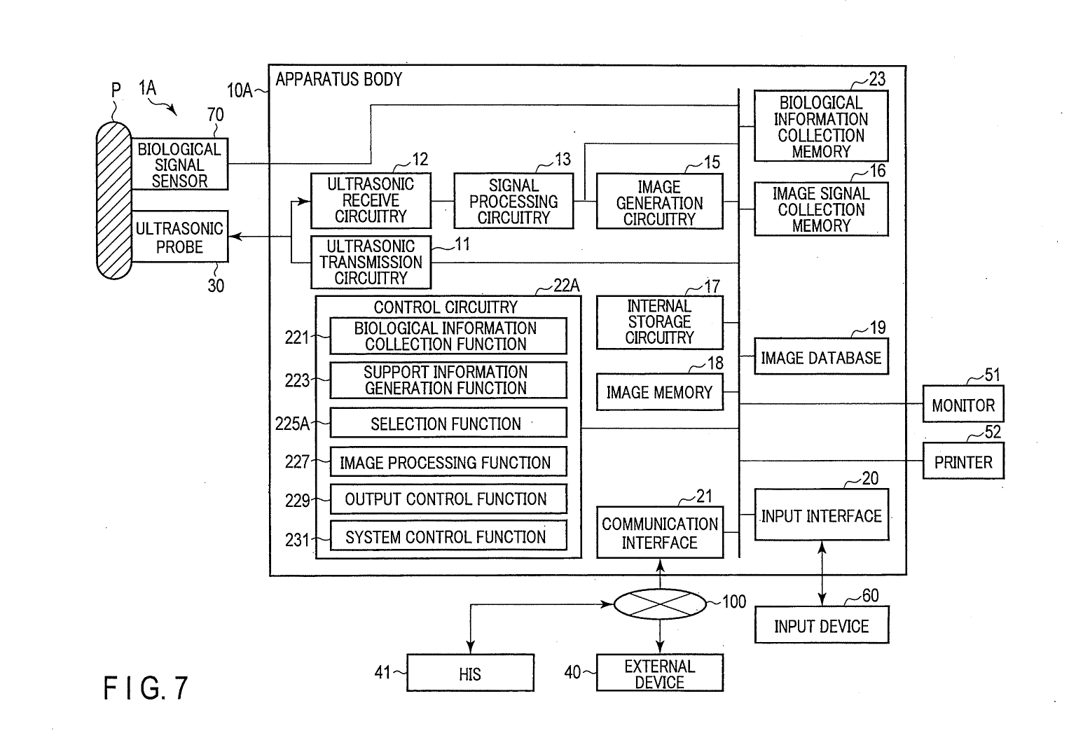

An ultrasonic diagnostic apparatus 1A according to the second embodiment will be explained with reference to the block diagram of FIG. 7.

As illustrated in FIG. 7, the ultrasonic diagnostic apparatus 1A includes an apparatus body 10A, the ultrasonic probe 30, the monitor 51, the printer 52, the input device 60, and the biological signal sensor 70. The apparatus body 10A is coupled to the external device 40 and the hospital information system (HIS) 41 via the network 500. The apparatus body 10A is also coupled to the monitor 51, the printer 52, and the input device 60.

The apparatus body 10A illustrated in FIG. 7 is configured to generate an ultrasonic image based on a reflection wave signal received by the ultrasonic probe 30. The apparatus body 10A includes, as illustrated in FIG. 7, the ultrasonic transmission circuitry 11, the ultrasonic receive circuitry 12, the signal processing circuitry 13, the image generation circuitry 15, the image signal collection memory 16, the internal storage circuitry 17, the image memory 18, the image database 19, the input interface 20, the communication interface 21, control circuitry 22A, and the biological information collection memory 23.

The control circuitry 22A may be a processor that serves as the center of the ultrasonic diagnostic apparatus LA. The control circuitry 22A executes an operation program stored in the internal storage circuitry 17, and thereby realizes functions corresponding to this operation program. Specifically, the control circuitry 22A is provided with a biological information collection function 221, a support information generation function 223, a selection function 225A, an image processing function 227, an output control function 229, and a system control function 231.

The selection function 225A is to select a heartbeat to be used for the ultrasonic image measurement and analysis, based on the ratio of the lengths of any consecutive heartbeats. When the selection function 225A is implemented, the control circuitry 22A selects consecutive heartbeats exhibiting the ratio of their lengths being approximately 1. For instance, the control circuitry 22A may select, based on the intervals between the R-wave peaks (hereinafter referred to as RR interval) of any consecutive ones of a plurality of heartbeats, the consecutive heartbeats that exhibit the ratio of their lengths being approximately 1. Specifically, the control circuitry 22A may select consecutive heartbeats in which the ratio of having the ratio of the first RR interval and the second RR interval immediately before the first RR interval is approximately 1. For the ratio, the first RR interval may be divided by the second RR interval. By defining “approximately 1”, a certain latitude is allowed for “1”, which may include any values between 0.99 and 1.01.

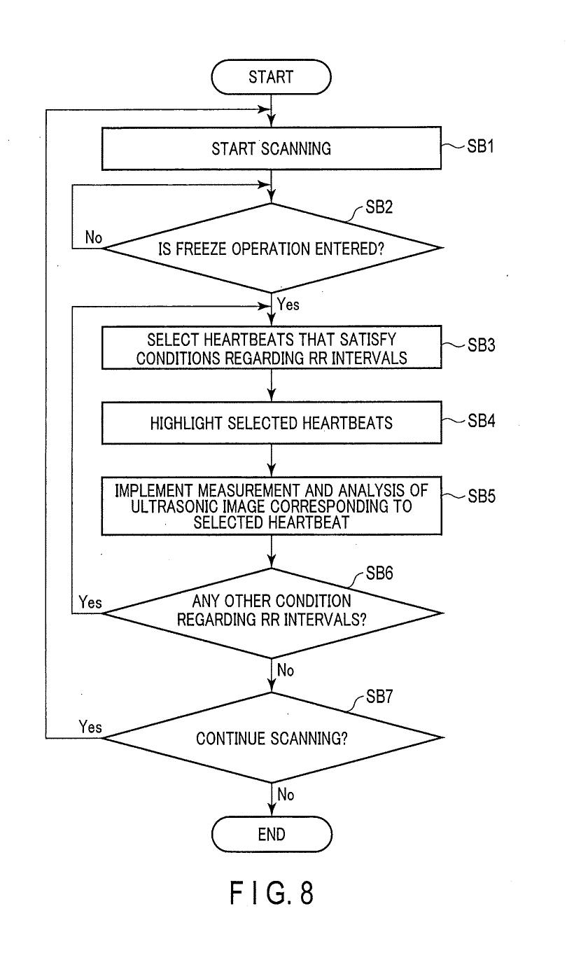

Next, the operation of the ultrasonic diagnostic apparatus 1A according to the second embodiment will be explained with reference to the drawings. FIG. 8 is a flowchart of the operation of the control circuitry 22A when the ultrasonic diagnostic apparatus 1A according to the second embodiment selects certain heartbeats. In the following explanation, the ultrasonic diagnostic apparatus 1A receives examination order information from the HIS 41, and implements the ultrasonic scanning based on the received examination order information. The control circuitry 22A collects heartbeat data output from the biological signal sensor 70 in accordance with ultrasonic scanning. Here, the control circuitry 22A associates the heartbeat data for every heartbeat with the respective items of the ultrasonic image data generated by the image generation circuitry 15, and sequentially stores the resultant data in the biological information collection memory 23. When implementing the ultrasonic scanning, the CW mode is adopted as an imaging mode.

The control circuitry 22A acquires in advance an RR interval-related condition for selecting heartbeats. The RR interval-related condition may be “heartbeats having the ratio of the first RR interval and the second RR interval being approximately 1 from among heartbeats based on the heartbeat data corresponding to a plurality of heartbeats collected during a predefined measurement duration”. Alternatively, the control circuitry 22A may acquire some other RR interval-related condition. If this is the case, the RR interval-related condition may be “heartbeats that are most frequently counted when heartbeats are counted according to specific RR intervals, based on the heartbeat data corresponding to a plurality of heartbeats collected for a predefined measurement duration”.

As illustrated in FIG. 8, when an start command for starting the ultrasonic scanning is entered via the input interface 20 after the examination order information is received from the HIS 41, the control circuitry 22A may control the ultrasonic transmission circuitry 11 and the ultrasonic receive circuitry 12 to start the ultrasonic scanning (step SB1).

The control circuitry 22A determines whether or not a freeze operation is entered (step SB2). When the control circuitry 22A determines that no freeze operation is entered (no at step SB2), the ultrasonic scanning is continued.

When determining that a freeze operation is entered (yes at step SB2), the control circuitry 22A may select, using the heartbeat data collected before this determination, heartbeats for the ultrasonic image measurement and analysis (step SB3). Specifically, the control circuitry 22A may select heartbeats having the ratio of the first RR interval and the second RR interval being approximately 1.

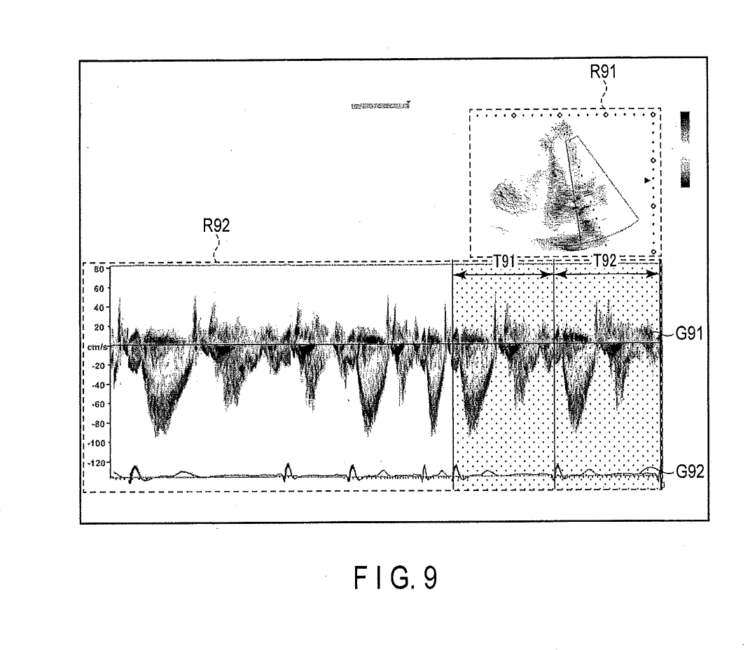

Next, the control circuitry 22A displays the selected heartbeats together with the ultrasonic images corresponding to these heartbeats. Here, the control circuitry 22A highlights the selected heartbeats (step SB4). Specifically, the control circuitry 22A generates highlight image data that represents a highlight image in which the selected heartbeats are emphasized. The control circuitry 22A superimposes a highlight image based on the generated highlight image data on the selected heartbeats, and displays the resultant image on the monitor 51. FIG. 9 is a diagram showing heartbeats displayed on the monitor by the ultrasonic diagnostic apparatus 1A according to the second embodiment. The heartbeats of FIG. 9 are displayed in the form of an electrocardiographic waveform. According to FIG. 9, a B-mode image region R91 and a Doppler image region R92 are displayed on the monitor 51.

As indicated in FIG. 9, the B-mode image region R91 includes a B-mode image to identify the scanning position for the ultrasonic scanning, for example, in the CW mode. The B-mode image region R91 may also include a color Doppler image superimposed on the B-mode image.

As indicated in FIG. 9, the Doppler image region R92 includes a Doppler spectrum image G91 acquired by the ultrasonic scanning. The Doppler image region R92 includes an electrocardiographic waveform G92 based on the heartbeat data that is associated with a Doppler spectrum image G91 with reference to the temporal axis. In the Doppler spectrum image G91 and the electrocardiographic waveform G92, highlight images are superimposed onto the regions for the consecutive two durations T91 and T92 corresponding to the selected heartbeats. The ratio of the durations T91 and T92 is approximately 1 in FIG. 9. In this manner, the regions included in the durations T91 and T92 are highlighted, which allows the operator to quickly identify the heartbeat suitable for the ultrasonic image measurement and analysis.

Next, the control circuitry 22A implements the measurement and analysis of an ultrasonic image corresponding to the heartbeat designated by the operator via the input interface 20, from among the heartbeats selected and displayed on the monitor 51 (step SB5). For example, the control circuitry 22 may implement the 2D WMT, 3D WMT, or auto EF. The control circuitry 22A may be configured to automatically implement the measurement and analysis on the ultrasonic image corresponding to the latest heartbeat from among the heartbeats selected at step SB3.

The control circuitry 22A determines whether or not there is any other RR interval-related condition (step SB6).

When determining that there is no other RR interval-related condition (no at step SB6), the control circuitry 22A determines whether the ultrasonic scanning should be continued (step SB7).

When determining that there is some other RR interval-related condition (yes at step SB6), the control circuitry 22A implements the operations of steps SB3 through SB6, based on this RR interval-related condition.

When no termination command to terminate the ultrasonic scanning is entered at step SB7 and thus it is determined that the ultrasonic scanning should be continued (yes at step SB7), the control circuitry 22A re-executes the operations at steps SB1 through SB7.

When a termination command to terminate the ultrasonic scanning is received, the control circuitry 22A determines that the ultrasonic scanning should not be continued (no at step SB7), and terminates the ultrasonic scanning.

According to the second embodiment, the control circuitry 22A collects the heartbeat data output from the biological signal sensor 70 in accordance with the ultrasonic scanning. The control circuitry 22 selects heartbeats to be used for the ultrasonic image measurement and analysis, based on the ratio of the lengths of consecutive heartbeats. The control circuitry 22A generates highlight image data that represents a highlight image in which the selected heartbeats are emphasized. The control circuitry 22A superimposes a highlight image based on the generated highlight image data, onto the selected heartbeats, and displays the resultant image on the monitor 51.

In this manner, the operator can quickly find a heartbeat for the ultrasonic image measurement and analysis, based on the heartbeat data. Thus, the trial and error time for the operator to select a target heartbeat can be reduced. Furthermore, because the selection is based on an objective selection condition, erroneous selection of heartbeat data can be avoided. In addition, consistency with the previous examination and consistency that is not affected by different operators can be maintained.

Thus, the examination efficiency can be enhanced.

### Modification Examples

In the second embodiment, the selection of a heartbeat for the ultrasonic image measurement and analysis based on the ratio of the lengths of consecutive heartbeats have been explained. The selection, however, is not limited to this explanation. In a modification example of the second embodiment, the selection of heartbeats for the ultrasonic image measurement and analysis with at least one of various heartbeat selection methods, including the heartbeat selecting method based on the ratio of the lengths of consecutive heartbeats, will be explained.

The structure of the ultrasonic diagnostic apparatus according to this modification is the same as the structure of the ultrasonic diagnostic apparatus 1A as illustrated in FIG. 7.

Next, the operation of selecting heartbeats for the ultrasonic image measurement and analysis with the selection function 225A according to the modification will be explained. When the selection function 225A is implemented, the control circuitry 22A adopts at least one of various heartbeat selection methods including the heartbeat selection method based on the ratio of the lengths of consecutive heartbeats in accordance with the designated heartbeat selection method, and selects heartbeats for the ultrasonic image measurement and analysis. That is, with the selection function 225A, the control circuitry 22 selects at least one of a plurality of heartbeats, based on a specific condition for selecting heartbeats. When multiple heartbeats that satisfy the condition are extracted, a heartbeat may be automatically selected at the control circuitry 22 from these heartbeats that satisfy the condition, or may be selected in response to a command entered by the operator through the input interface 20.

The control circuitry 22A may select a heartbeat having approximately the same length as a heartbeat obtained based on a heart rate counted at a previous ultrasonic examination of the same patient. By specifying a “heartbeat having approximately the same length”, when the heart rate is 60 and the length of heartbeat obtained based on the heart rate is 1000 (msec), any heartbeat having a length of 1000 (msec) and also in its deviation of, for example 1000 (msec)±100 (msec), is displayed.

The control circuitry 22A may select a heartbeat having approximately the same length as a heartbeat obtained based on the heart rate counted in an examination of the same patient, using any medical image diagnostic apparatus other than the ultrasonic diagnostic apparatus. Examples of other medical image diagnostic apparatuses may include an X-ray CT apparatus, MRI apparatus, nuclear medicine diagnostic apparatus, and X-ray diagnostic apparatus. For the heart rate counted at the examination using a different medical image diagnostic apparatus, the operator may enter its value through the input interface 20.

The control circuitry 22A may select a heartbeat having approximately the same length as a heartbeat obtained based on the heart rate calculated from moving averages during the ultrasonic scanning. The heart rate calculated from the moving averages may be displayed at a predetermined position of the display screen on the monitor 51 during the ultrasonic scanning.

The control circuitry 22A may select a heartbeat using artificial intelligence (AI). Specifically, the control circuitry 22A uses a heartbeat selection program for selecting a heartbeat, which is implemented in the HIS 41. This heartbeat selection program incorporates an identifier, which is generated as a result of machine learning such as deep learning using the heartbeat data accumulated in a medical institution, for example, in association with patient information including their ages, sexes, and case history. The identifier may be constituted by a function defining an optimized neural network and optimized parameters. The heartbeat selection program may be implemented in the ultrasonic diagnostic apparatus 1A.

The control circuitry 22A removes any heartbeat having a length shorter than or equal to a predefined length, for example shorter than or equal to 300 (msec). With any heartbeats shorter than or equal to 300 (msec) removed, heartbeats having a length greater than 300 (msec) can be selected.

In light of the above, the conditions for selecting a specific heartbeat include at least one of “selecting consecutive heartbeats having the ratio of their lengths being approximately 1”, “selecting a heartbeat having approximately the same length as a heartbeat based on the heart rate counted in the previous ultrasonic examination”, “selecting a heartbeat using an identifier generated as a result of certain machine learning”, “selecting a heartbeat having approximately the same length as the heartbeat obtained based on the heart rate counted at an examination using a different medical image diagnostic apparatus” and “selecting a heartbeat having approximately the same length as a designated heartbeat”. The conditions for selecting a heartbeat may further include “removing a heartbeat of a length shorter than or equal to a predefined length from selection targets”.

The control circuitry 22 may select, with the selection function 225A, any of the conditions for selecting a desired heartbeat. If this is the case, the operator selects a condition from among the conditions displayed on the monitor 51 via the input interface 20. Upon receipt of the condition selected by the operator, the control circuitry 22 determines the condition for selecting a heartbeat.

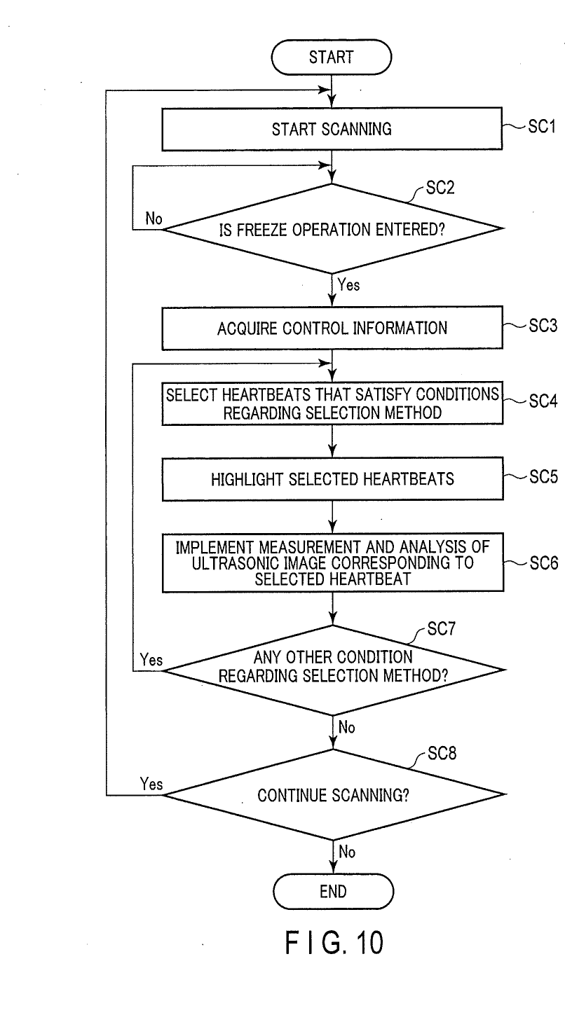

Next, the operation of the ultrasonic diagnostic apparatus 1A according to a modification example will be explained with reference to FIG. 10. A flowchart of the operation of the control circuitry 22A when the ultrasonic diagnostic apparatus 1A according to a modification example of the second embodiment selects a desired heartbeat is presented in FIG. 10. In the following explanation, the ultrasonic diagnostic apparatus 1A receives examination order information from the HIS 41, and implements the ultrasonic scanning based on the received examination order information. The examination order information may contain patient information including age, sex, case history, etc. The control circuitry 22A collects heartbeat data output from the biological signal sensor 70 in accordance with the ultrasonic scanning. Here, the control circuitry 22A associates the heartbeat data for every heartbeat with the respective items of the ultrasonic image data generated by the image generation circuitry 15 and sequentially stores the resultant data in the biological information collection memory 23.

The operations at steps SC1 and SC2 in FIG. 10 are the same as the operations at steps SB1 and SB2 in FIG. 8.

When determining that a freeze operation is entered (yes at step SC2), the control circuitry 22A acquires control information for selecting a heartbeat (step SC3). The control circuitry 22A may acquire, via the input interface 20, control information including conditions related to the heartbeat selection method that indicates which of the selection methods should be adopted for selection of a heartbeat. According to the modification example of the second embodiment, the condition related to the heartbeat selection method is “selecting a heartbeat based on input of the patient's information, using an artificial intelligence technique”. The control information may contain a plurality of conditions regarding the heartbeat selection method. Other conditions regarding the heartbeat selection method may be “selecting a heartbeat having approximately the same length as the length of a heartbeat based on the heart rate of the same patient counted in the previous ultrasonic examination”, “selecting a heartbeat having approximately the same length as the length of a heartbeat based on the heart rate of the same patient counted at an examination using an MRI apparatus”, “removing heartbeats of 300 milliseconds or shorter from the selection targets”, and “selecting heartbeats including the ratio of the first RR interval and the second RR interval being approximately 1 based on the heartbeats corresponding to the heartbeat data collected during a predefined measurement duration”. Furthermore, the control circuitry 22A may read the prestored control information from the internal storage circuitry 17 to acquire the control information.

The control circuitry 22A selects a heartbeat in accordance with the condition regarding the heartbeat selection method which is contained in the acquired control information, using the heartbeat data collected until the input of a freeze operation is determined (step SC4). The control circuitry 22A selects a heartbeat based on the condition regarding the heartbeat selection method, “selecting a heartbeat based on the input patient information, using an artificial intelligence technique”. Specifically, the control circuitry 22A enters patient information including age, sex, and case history, such as 60 years old, female, and cardiac infraction, on the heartbeat selection program implemented in the HIS 41 via the communication interface 21.

When the patient information is entered, the heartbeat selection program implemented in the HIS 41 may apply the entered patient information to the identifier, and may have the identifier output the heartbeat data for the ultrasonic image measurement and analysis. The heartbeat selection program transmits, via the communication interface 21, the output heartbeat data to the control circuitry 22A of the ultrasonic diagnostic apparatus 1A.

The control circuitry 22A receives the heartbeat data that is transmitted from the heartbeat selection program implemented in the HIS 41, and selects a heartbeat for the ultrasonic image measurement and analysis, based on the received heartbeat data. The control circuitry 22A may select a heartbeat having approximately the same length as the length corresponding to one heartbeat, based on the received heartbeat data. In this manner, a heartbeat for the ultrasonic image measurement and analysis can be selected in consideration of the conditions of individual patients. The control circuitry 22A may select a heartbeat having a waveform similar to one heartbeat based on the received heartbeat data.

The operations at steps SC5 and SC6 in FIG. 10 are the same as the operations at steps SB4 and SB5 in FIG. 8.

After executing step SC6, the control circuitry 22A determines whether there is any other condition regarding the heartbeat selection method (step SC7).

When determining that there is no other condition regarding the heartbeat selection method (no at step SC7), the control circuitry 22A determines whether or not to continue the ultrasonic scanning (step SC8).

When determining that there is some other condition regarding the heartbeat selection method (yes at step SC7), the control circuitry 22A executes the operations from steps SC4 to SC7 based on this condition regarding the heartbeat selection method.

At step SC8, if the control circuitry 22A determines that the ultrasonic scanning should be continued in the absence of a termination command for terminating the ultrasonic scanning (yes at step SC8), the control circuitry 22A repeats the operations from steps SC1 to SC8.

When a termination command to terminate the ultrasonic scanning is received, the control circuitry 22A determines that the ultrasonic scanning should not be continued (no at step SC8), and terminates the ultrasonic scanning.

According to the modification of the second embodiment, the control circuitry 22A selects a heartbeat in accordance with the designated one of the heartbeat selection methods. In this manner, the most suitable heartbeat can be selected for the ultrasonic image measurement and analysis.

### Other Embodiments

According to the first embodiment, the ultrasonic diagnostic apparatus 1 highlights the selected heartbeats for the display. However, the configuration is not limited thereto. The control circuitry 22 of the ultrasonic diagnostic apparatus 1 may display on the monitor 51 an electrocardiographic waveform based on the heartbeat data corresponding to a plurality of heartbeats. The control circuitry 22 receives via the input interface 20 the designated heartbeat for the displayed electrocardiographic waveform. The control circuitry 22 highlights the heartbeats having approximately the same length as the designated heartbeat.

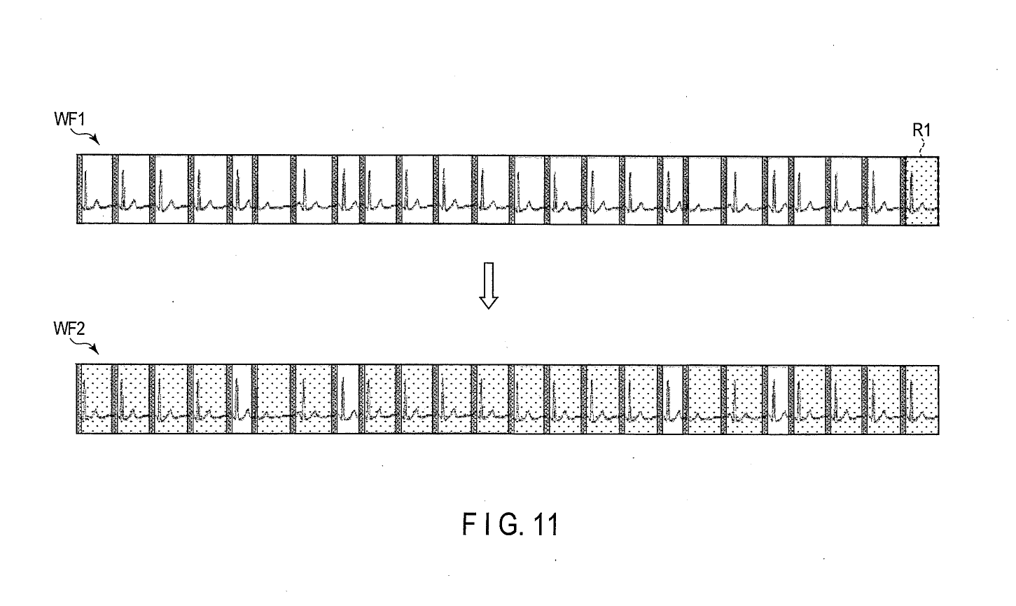

FIG. 11 is a diagram explaining a display mode of heartbeats that an ultrasonic diagnostic apparatus 1 according to another embodiment displays on the monitor 51. The heartbeats in FIG. 11 are displayed as an electrocardiographic waveform. According to FIG. 11, the control circuitry 22 of the ultrasonic diagnostic apparatus 1 first displays an electrocardiogram WF1 on the monitor 51. When a region R1 corresponding to one heartbeat is designated from the electrocardiogram WF1, the control circuitry 22 displays on the monitor 51 an electrocardiogram WF2 in which any heartbeats having approximately the same length as the length of the heartbeat corresponding to the region R1 are highlighted. In the electrocardiogram WF2 of FIG. 11, the highlighted regions are provided with a hatch pattern. Specifically, the control circuitry 22 generates highlight image data representing a highlight image in which any heartbeats, having approximately the same length as the length of the electrocardiographic waveform corresponding to the region R1, are emphasized. The control circuitry 22 superimposes the highlight image based on the highlight image data onto the electrocardiogram WF1, and displays the resultant image as the electrocardiogram WF2. In this manner, the operator is allowed to select a heartbeat having a desired length.

Furthermore, according to the modification example of the second embodiment, the identifier of the heartbeat selection program has learned to use patient information including age, sex, and case history of individual patients and to output the heartbeat data corresponding to a specific patient from the entered information, but the example is not limited thereto. The identifier may learn to use patient information including the age, sex, and case history of individual patients and to output a selection method. If this is the case, the control circuitry 22A adopts only “selecting a heartbeat based on the entered patient information, using the artificial intelligence technique” as a condition regarding the heartbeat selection method. In this manner, the trial and error time for the operator to determine a selection method can be reduced.

The above “processor” may denote any circuitry such as a central processing unit (CPU), graphics processing unit (GPU), application specific integrated circuit (ASIC), or programmable logic device (e.g., simple programmable logic device (SPLD), complex programmable logic device (CPLD), and field programmable gate array (FPGA)). The processor realizes the functions by reading and implementing the programs stored in the storage circuitry. The processors according to the embodiments of the present invention are not limited to a single circuit for each processor, but may be configured as a processor by combining different independent circuits to realize the functions. Each processor of the present embodiments is not limited to be configured as single circuitry, but may include a plurality of units of independent circuitry, in order to implement the functions. Furthermore, a plurality of constituent elements shown in FIGS. 1, and 7 may be integrated into one processor to implement the functions.

According to at least one of the above embodiments, the examination efficiency can be enhanced.

While certain embodiments have been described, these embodiments have been presented by way of example only, and are not intended to limit the scope of the inventions. Indeed, the novel embodiments described herein may be embodied in a variety of other forms. Furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An ultrasonic diagnostic apparatus, comprising:
processing circuitry configured to:
select at least one heartbeat from among a plurality of heartbeats based on a heartbeat selection condition;
generate a highlight image in which a range corresponding to the selected heartbeat is emphasized; and
display an electrocardiographic waveform corresponding to the heartbeats, an ultrasonic image corresponding to the electrocardiographic waveform, and the highlight image.

2. The ultrasonic diagnostic apparatus according to claim 1, wherein:
the ultrasonic image is a B-mode image or a Doppler spectrum image.

3. The ultrasonic diagnostic apparatus according to claim 1, wherein:
the ultrasonic image is a Doppler spectrum image, and
the processing circuitry is further configured to:
superimpose the highlight image on the electrocardiographic waveform and the Doppler spectrum image, and display the superimposed image.

4. The ultrasonic diagnostic apparatus according to claim 1, wherein:
the heartbeat selection condition includes selecting consecutive heartbeats having a ratio of lengths of the heartbeats being approximately 1.

5. The ultrasonic diagnostic apparatus according to claim 1, wherein:
the heartbeat selection condition includes selecting a heartbeat having a length approximately same as a length of a heartbeat based on a heart rate counted in a previous ultrasonic examination.

6. The ultrasonic diagnostic apparatus according to claim 1, wherein:
the heartbeat selection condition includes selecting the heartbeat, using an identifier generated as a result of predetermined machine learning.

7. The ultrasonic diagnostic apparatus according to claim 1, wherein:
the heartbeat selection condition includes selecting a heartbeat having a length approximately same as a length of a heartbeat based on a heart rate counted in an examination employing a medical image diagnostic apparatus different from the ultrasonic diagnostic apparatus.

8. The ultrasonic diagnostic apparatus according to claim 1, wherein:
the heartbeat selection condition includes removing a heartbeat having a length shorter than or equal to a predetermined length, from selection targets.

9. The ultrasonic diagnostic apparatus according to claim 1, wherein:
the heartbeat selection condition includes selecting a heartbeat having a length approximately same as a designated length of a heartbeat.

10. An ultrasonic diagnostic apparatus, comprising:
processing circuitry configured to:
acquire an ultrasonic image of a subject;
acquire heartbeat data of a plurality of heartbeats of the subject for a duration of acquiring the ultrasonic image;
generate support information for supporting heartbeat selection based on the heartbeat data; and
display the support information, an electrocardiographic waveform based on the heartbeat data, and the ultrasonic image corresponding to the electrocardiographic waveform.

11. The ultrasonic diagnostic apparatus according to claim 10, wherein:
the support information is a heartbeat histogram representing a relationship between a length of a heartbeat and the number of heartbeats counted according to lengths of the heartbeats,
the processing circuitry is further configured to:
generate the heartbeat histogram using the heartbeat data;
select at least one heartbeat based on the heartbeat histogram;
generate a highlight image in which a range corresponding to the selected heartbeat is emphasized; and
superimpose the highlight image on the electrocardiographic waveform and the ultrasonic image corresponding to the heartbeats, and display the superimposed image.

12. The ultrasonic diagnostic apparatus according to claim 10, wherein:
the support information is a heartbeat histogram representing a relationship between a length of one heartbeat and the number of heartbeats counted according to lengths of the heartbeats, and
the processing circuitry is further configured to:
generate the heartbeat histogram using the heartbeat data;
generate, based on the heartbeat histogram, a highlight image in which a range corresponding to at least one selected heartbeat is emphasized; and
superimpose the highlight image on the electrocardiographic waveform and the ultrasonic image of the heartbeat.

13. The ultrasonic diagnostic apparatus according to claim 10, wherein:
the support information is an electrocardiographic waveform in which consecutive heartbeats having a ratio of lengths being approximately 1 are highlighted, and
the processing circuitry is further configured to:
generate the electrocardiographic waveform using the heartbeat data;
select at least one heartbeat based on the electrocardiographic waveform;
generate a highlight image in which a range corresponding to the selected heartbeat is emphasized; and
superimpose the highlight image on the electrocardiographic waveform and the ultrasonic image of the heartbeat and display the superimposed image.

14. The ultrasonic diagnostic apparatus according to claim 10, wherein:
the support information is an electrocardiographic waveform in which a heartbeat having a length approximately same as a length of a heartbeat based on a heart rate counted in a previous ultrasonic examination is highlighted, and
the processing circuitry is further configured to:
generate the electrocardiographic waveform using the heartbeat data;
select at least one heartbeat based on the electrocardiographic waveform;
generate a highlight image in which a range corresponding to the selected heartbeat is emphasized; and
superimpose the highlight image on the electrocardiographic waveform and the ultrasonic image of the heartbeat, and display the superimposed image.

15. The ultrasonic diagnostic apparatus according to claim 10, wherein:
the support information is an electrocardiographic waveform in which a heartbeat having a length approximately same as a length of a heartbeat based on a heart rate counted in an examination employing a medical image diagnostic apparatus different from the ultrasonic diagnostic apparatus is highlighted, and
the processing circuitry is further configured to:
generate the electrocardiographic waveform using the heartbeat data;
select at least one heartbeat based on the electrocardiographic waveform;
generate a highlight image in which a range corresponding to the selected heartbeat is emphasized; and
superimpose the highlight image on the electrocardiographic waveform and the ultrasonic image of the heartbeat and display the superimposed image.

16. The ultrasonic diagnostic apparatus according to claim 10, wherein:
the support information is an electrocardiographic waveform in which a heartbeat having a length approximately same as a length of a designated heartbeat is highlighted, and
the processing circuitry is further configured to:
generate the electrocardiographic waveform using the heartbeat data;
select at least one heartbeat based on the electrocardiographic waveform;
generate a highlight image in which a range corresponding to the selected heartbeat is emphasized; and
superimpose the highlight image on the electrocardiographic waveform and the ultrasonic image of the heartbeat, and display the superimposed image.
